(19) United States
(12) Patent Application Publication
(10) Pub. No.: US 2012/0130134 A1
(43) Pub. Date: May 24, 2012

(54) PROCESS FOR THE PREPARATION OF POLYETHEROLS FROM ALKYLENE OXIDES

(75) Inventors: Matthias Schopohl, Dresden (DE); Thomas Ostrowski, Mannheim (DE); Dirk Meckelnburg, Limburgerhof (DE); Renata Jovanovic, Ludwigshafen (DE); Achim Loeffler, Speyer (DE); Hans-Juergen Pallasch, Kallstadt (DE); Thorsten Friese, Mannheim (DE); Eckard Schauss, Heuchelheim (DE); Susanne Szeschkus, Pfungstadt (DE); Vinit Chilekar, Mannheim (DE); Hans Volkmar Schwarz, Ludwigshafen (DE)

(73) Assignee: BASF SE, Ludwigshafen (DE)

(21) Appl. No.: 13/387,540

(22) PCT Filed: Jul. 27, 2010
(86) PCT No.: PCT/EP10/60845

§ 371 Date: Jan. 27, 2012

(30) Foreign Application Priority Data
Jul. 29, 2009 (EP) 09166741.0

(51) Int. Cl. C07C 29/04

(52) U.S. Cl. 568/896

(57) ABSTRACT

A process for the preparation of polyetherols, comprising the following steps:
a) reaction of at least one starter compound with at least one alkylene oxide and a catalyst to give a crude polyetherol and
b) removal of low molecular weight byproducts from the crude polyetherol from step a), wherein the crude polyetherol is treated with a stripping gas in a column having internals.

Polyetherols are used, for example, for the preparation of polyurethane plastics, such as polyurethane foams, polyurethane cast skins and elastomers, which in turn are used for producing moldings, such as mattresses, cushions, articles of upholstered furniture and components for the automotive industry. For this reason, there is a need for polyetherols which are free of toxic substances and have no troublesome odors. Troublesome odors and toxic substances are undesired in polyurethane preparation.

The troublesome odors are caused by low molecular weight compounds, such as unreacted alkylene oxides, and monofunctional byproducts.

Processes for the preparation of polyetherols are known and are described in the prior art. Polyetherols are prepared by polyaddition of alkylene oxides with polyfunctional starter compounds. Starter compounds used are, for example, water, alcohols, acids and amines or mixtures of these compounds. Catalysts used for the addition reaction are alkali metal hydroxide catalysts, amine catalysts and multimetal cyanide catalysts (DMC catalysts). With the use of alkali metal hydroxide catalysts, the direct use of the polyetherols obtained for the preparation of polyurethanes is not possible since the crude polyetherols obtained in this manner have a reactivity which is too high. For this reason, a neutralization of the crude polyetherols with dilute acid for eliminating the alkaline catalyst has to be carried out for working up in the preparation of polyetherols using alkali metal hydroxide catalysts. Since salts form in the neutralization, these must be separated from the polyetherols in a further working-up step by filtration or extraction. In the synthesis described above, low molecular weight byproducts or residual monomers which are toxic and/or have an intense odor are converted into low-odor and nontoxic substances by the neutralization with dilute acid. Owing to the multiplicity of working-up steps necessary, such as neutralization and removal of the salts, the alkali metal hydroxide-catalyzed preparation of polyetherols is technically complicated.

The processes described in the prior art in which amine or DMC catalysts are used have the advantage over the alkali metal hydroxide-catalyzed processes that, in them, the catalyst used can remain in the product and a neutralization associated with subsequent removal of the resulting salts is unnecessary. However, this process has the disadvantage that the crude polyetherol obtained still comprises low molecular weight compounds, such as toxic alkylene oxides and monofunctional byproducts, which are harmful to health or lead to troublesome odors. The crude polyetherols thus obtained are unsuitable for the preparation of polyurethanes, especially owing to the toxic alkylene oxides.

For working up polyetherols which are prepared by amine or DMC catalysis, the prior art describes processes in which the crude polyetherol is treated batchwise with steam or nitrogen.

EP 1 756 198 describes a process for the preparation of low-odor polyetherols using a DMC catalyst, in which, for removing low molecular weight byproducts, the crude polyetherol is treated with nitrogen in a stirred reactor in a batchwise process.

DE 103 24 998 likewise describes a process for the preparation of low-odor polyetherols using a DMC catalyst. The low molecular weight byproducts are separated off in a batchwise process by treating the crude polyetherol with steam or a mixture of steam and nitrogen with the aid of a pure or of a stirred bubble column.

The batchwise processes described in the prior art for the preparation of polyetherols comprise, for removing low molecular weight byproducts from the crude polyetherol, steps which require separation units which are complicated in terms of apparatus, such as bubble columns, stirred bubble columns or stirred reactors. These separation units are operated only batchwise since they have only limited suitability for continuous operation.

The object of the present invention is to provide a process for the preparation of polyetherols which permits commercial and economical removal of low molecular weight byproducts from crude polyetherols. A further object of the present invention is to provide a continuous process for the preparation of polyetherols, in which the removal of low molecular weight byproducts from crude polyetherols can be operated in a continuous procedure.

According to the invention, this object is achieved by a process for the preparation of polyetherols in which the removal of the low molecular weight byproducts from the crude polyetherols is effected with a stripping gas in a column having internals.

The present invention therefore relates to a process for the preparation of polyetherols, comprising the following steps:
a) reaction of at least one starter compound with at least one alkylene oxide and a catalyst to give a crude polyetherol and
b) removal of low molecular weight byproducts from the crude polyetherol from step a),
   wherein the crude polyetherol is treated in step b) with a stripping gas in a column having internals.

The invention furthermore relates to polyetherols obtainable by the process according to the invention, and to the use of the polyetherols for the synthesis of polyurethanes.

Compared with the processes described in the prior art, the process according to the invention has increased efficiency and moreover can be operated in a continuous procedure. The process according to the invention can moreover be operated at lower pressures than the process described in the prior art, with the result that the removal of volatile constituents is improved. In addition, in some cases, the removal can be effected in substantially smaller reactor volumes by the process according to the invention compared with the prior art.

The invention therefore also relates to a process for the preparation of polyetherols in which the preparation of the crude polyetherol (step a)) and the removal of low molecular weight byproducts from the crude polyetherol (step b)) is effected with a stripping gas in a column having internals in a continuous procedure.

In the context of the present invention, crude polyetherols are understood as meaning polyetherols which are prepared by one of the processes described above in step a) and comprise impurities, such as low molecular weight byproducts. Low molecular weight byproducts are understood as meaning alkylene oxides, such as ethylene oxide and propylene oxide, and other byproducts having a low functionality, such as, for example, aldehydes and ketones, which either form as byproducts during the alkoxylation or enter the polyol as impurities via the alkylene oxides or starter compounds and have troublesome odors.

The preparation of the crude polyetherol in step a) is known per se and is described in the prior art. The preparation of the crude polyetherol can be effected by means of alkali metal hydroxide, amine or DMC catalysis. Preferably, the crude polyetherols are prepared by amine- or DMC-catalyzed reactions.

The chemical and physical properties of the crude polyetherols prepared may vary within wide ranges. Preferably, crude polyetherols for the production of rigid foam polyetherols having an average molecular weight of ≦1500 g/mol or the production of flexible foam polyetherols or CASE polyetherols (coatings, adhesives, sealants, elastomers) having an average molecular weight of ≦20 000 g/mol, which comprise ethylene oxide and/or propylene oxide units, are prepared in step a).

The crude polyetherols prepared in step a) are preferably used in fresh form in step b). In the context of the present invention, freshly prepared crude polyetherols are understood as meaning polyetherols which were produced no longer than 12 hours beforehand, preferably no longer than 6 hours, more preferably no longer than 3 hours and particularly preferably no longer than 30 minutes beforehand. In a further particularly preferred embodiment, the crude polyetherol prepared in step a) is used directly in the purification step (step b)).

In a particular embodiment, the crude polyetherol is prepared in a continuous process in step a). The preparation of the crude polyetherol by amine- and DMC-catalyzed continuous processes is particularly preferred.

For the preparation of flexible foam polyetherols, DMC-catalyzed continuous processes are particularly preferred. For the preparation of rigid foam polyetherols, amine-catalyzed processes are particularly preferred.

For the preparation of flexible foam polyetherols, step a) can be carried out, for example, according to EP 1 763 550. The continuous preparation of the crude polyetherol in step a) is effected by an addition reaction of alkylene oxides with H-functional starter substances with the use of a DMC catalyst, comprising the steps:
i) preparation of a crude polyetherol by continuous metering of at least one starter compound, an alkylene oxide or a mixture of at least two alkylene oxides and the required amount of DMC catalyst in a continuous reactor,
ii) continuous removal of the product from step i) from the reactor.

In a further embodiment, it is also possible in step a) to prepare crude polyetherols which comprise two segments having different compositions in the polyether chain. These are obtainable, for example, in step a) by the continuous preparation of polyetherols by an addition reaction of alkylene oxides with starter compounds with the use of a DMC catalyst, comprising the steps:
i) preparation of a crude polyetherol by continuous metering of at least one starter compound, an alkylene oxide or a mixture of at least two alkylene oxides and the required amount of DMC catalyst in a continuous reactor, and
ii) continuous removal of the crude polyetherol from step i) from the reactor and optionally
iii) continuous metering of the crude polyetherol from step i), of an alkylene oxide differing from that in step i) or of a mixture of at least two alkylene oxides which differs from the mixture in step i) and optionally the required amount of DMC catalyst into a further continuous reactor,
iv) continuous removal of the block polyetherol from step iii) from the reactor.

The continuous preparation of rigid foam polyetherols can be effected, for example, analogously to the continuous process for the preparation of flexible foam polyetherols, an amine catalyst being used instead of the DMC catalyst. The preparation of rigid foam polyetherols is also described in WO 2007/147780.

All compounds which have an active hydrogen are suitable as the starter compound. According to the invention, OH-functional compounds are preferred as starter compounds.

According to the invention, compounds having 2-8 functional groups with acidic hydrogen atoms, such as polyalcohols or polyamines, are suitable as starter compounds for the preparation of flexible foam polyetherols. For example, the following compounds are suitable: water, organic dicarboxylic acids, such as succinic acid, adipic acid, phthalic acid and terephthalic acid, and mono- and polyhydric alcohols, such as monoethylene glycol, 1,2- and 1,3-propanediol, diethylene glycol, dipropylene glycol, 1,4-butanediol, 1,6-hexanediol, glycerol, trimethylolpropane, pentaerythritol, sorbitol and sucrose. Adducts of ethylene oxide and/or propylene oxide with water, monoethylene glycol, diethylene glycol, 1,2-propanediol, dipropylene glycol, glycerol, trimethylolpropane, amines, such as triethanolamine, tri(2-propanolamine), tri(3-propanolamine), ethylenediamine, propylenediamine, vicinal toluenediamine, 2,6- or 2,4-substituted toluenediamine, diphenylmethanediamine, pentaerythritol, sorbitol and/or sucrose, individually or as mixtures, are preferably used as polyether polyalcohols. For the preparation of flexible foam polyetherols, compounds which have 2 to 4 reactive (acidic) hydrogen atoms are preferred.

According to the invention, the starter compounds may also be used in the form of alkoxylates. In particular, alkoxylates having a molecular weight M_{w} in the range from 62 to 15 000 g/mol are preferred.

Also suitable as starter compounds for flexible foams are macromolecules having functional groups which have active hydrogen atoms, for example hydroxyl groups, in particular those which are mentioned in WO 01/16209.

In addition to the compounds mentioned above under the flexible foam polyetherols, starter compounds having amino groups can also be used as a starter compound for the preparation of rigid foam polyetherols. For the preparation of rigid foam polyetherols, starter substances having at least 3 reactive hydrogen atoms are preferably used. They are preferably aliphatic amines, in particular ethylenediamine, and aromatic amines, in particular toluenediamine (TDA) and mixtures of isomers of diphenylmethane diisocyanate and its higher homologs (MDA), mixtures of aromatic and aliphatic amines or solid OH-functional compounds, such as pentaerythritol, carbohydrates, preferably starch, cellulose and particularly preferably sugars, in particular sorbitol, mannitol, glucose, fructose and sucrose. The use of melamine and its H-functional derivatives is also possible.

In principle, all suitable alkylene oxides can be used for the process according to the invention. For example, C₂-C₂₀-alkylene oxides, such as, for example, ethylene oxide, propylene oxide, 1,2-butylene oxide, 2,3-butylene oxide, isobutylene oxide, pentene oxide, hexene oxide, cyclohexene oxide, styrene oxide, dodecene epoxide, octadecene epoxide, and mixtures of these epoxides are suitable. Ethylene oxide, propylene oxide, 1,2-butylene oxide, 2,3-butylene oxide, isobutylene oxide and pentene oxide are particularly suitable, propylene oxide and ethylene oxide being particularly preferred.

The DMC-catalyzed process is described in more detail below. In principle, all suitable compounds known to the person skilled in the art can be used as a DMC catalyst.

DMC compounds suitable as a catalyst are described, for example, in WO 99/16775 and DE 10117273.7. In particular, double metal cyanide compounds of the general formula I are suitable as catalyst for the alkoxylation:

M¹ₐ[M²(CN)_{b}(A)_{c}]_{d}.fM¹_{g}Xₙ.h(H₂O).eL.kP (I),

in which
M¹ is at least one metal ion selected from the group consisting of Zn²⁺, Fe²⁺, Fe³⁺, Co³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W⁴⁺, W⁶⁺, Cr²⁺, Cr³⁺, Cd²⁺, Hg²⁺, Pd²⁺, Pt²⁺, V²⁺, Mg²⁺, Ca²⁺, Ba²⁺, Cu²⁺, La³⁺, Ce³⁺, Ce⁴⁺, Eu³⁺, Ti³⁺, Ti⁴⁺, Ag⁺, Rh²⁺, Rh³⁺, Ru²⁺, Ru³⁺,
M² is at least one metal ion selected from the group consisting of Fe²⁺, Fe³⁺, Co²⁺, Co³⁺, Mn²⁺, Mn³⁺, V⁴⁺, V⁵⁺, Cr²⁺, Cr³⁺, Rh³⁺, Ru²⁺, Ir³⁺,
A and X, independently of one another, are an anion selected from the group consisting of halide, hydroxide, sulfate, carbonate, cyanide, thiocyanate, isocyanate, cyanate, carboxylate, oxalate, nitrate, nitrosyl, hydrogen sulfate, phosphate, dihydrogen phosphate, hydrogen phosphate or bicarbonate,
L is a water-miscible ligand selected from the group consisting of alcohols, aldehydes, ketones, ethers, polyethers, esters, polyesters, polycarbonate, ureas, amides, primary, secondary and tertiary amines, ligands comprising pyridine nitrogen, nitriles, sulfides, phosphides, phosphites, phosphanes, phosphonates and phosphates,
k is a fraction or integer greater than or equal to zero, and
P is an organic additive,
a, b, c, d, g and n are selected so that the electroneutrality of the compound (I) is ensured, it being possible for c to be 0,
e is the number of ligand molecules and is a fraction or integer greater than 0 or is 0,
f, h and m, independently of one another, are a fraction or integer greater than 0 or are 0.

The following may be mentioned as organic additives P: polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyalkyl acrylates, polyalkyl methacrylates, polyvinyl methyl ether, polyvinyl ethyl ether, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkyleneimines, maleic acid and maleic anhydride copolymers, hydroxyethylcellulose, polyacetates, ionic surface-active and interface-active compounds, gallic acid or its salts, esters or amides, carboxylic esters of polyhydric alcohols and glycosides.

These catalysts may be crystalline or amorphous. Where k is equal to zero, crystalline double metal cyanide compounds are preferred. Where k is greater than zero, crystalline, semicrystalline and substantially amorphous catalysts are preferred.

There are various preferred embodiments of the modified catalysts. One preferred embodiment comprises catalysts of the formula (I) in which k is greater than zero. The preferred catalyst then comprises at least one double metal cyanide compound, at least one organic ligand and at least one organic additive P.

In the case of another preferred embodiment, k is equal to zero, optionally e is also equal to zero and X is exclusively a carboxylate, preferably formate, acetate and propionate. Such catalysts are described in WO 99/16775. In this embodiment, crystalline double metal cyanide catalysts are preferred. Double metal cyanide catalysts as described in WO 00/74845, which are crystalline and lamellar, are furthermore preferred.

The modified catalysts are prepared by combining a metal salt solution with a cyanometallate solution which may optionally comprise both an organic ligand L and an organic additive P. Thereafter, the organic ligand and optionally the organic additive are added. In a preferred embodiment of the catalyst preparation, an inactive double metal cyanide phase is first prepared and is then converted into an active double metal cyanide phase by recrystallization, as described in PCT/EP01/01893.

In another preferred embodiment of the catalysts, f, e and k are not equal to zero. These are double metal cyanide catalysts which comprise a water-miscible organic ligand (in general in amounts of from 0.5 to 30% by weight) and an organic additive (in general in amounts of from 5 to 80% by weight), as described in WO 98/06312. The catalysts can be prepared either with vigorous stirring (24 000 rpm with Turrax) or with stirring, as described in U.S. Pat. No. 5,158,922.

In particular, double metal cyanide compounds which comprise zinc and cobalt or iron and cobalt are suitable as a catalyst for the alkoxylation. Zinc-cobalt catalysts are particularly suitable.

Crystalline DMC compounds are preferably used. In one preferred embodiment, a crystalline DMC compound of the Zn-Co type, which comprises zinc acetate as a further metal salt component, is used as a catalyst. Such compounds crystallize with a monoclinic structure and have a lamellar habit. Such compounds are described, for example, in WO 00/74845 or PCT/EP01/01893,

DMC compounds suitable as a catalyst can in principle be prepared by all methods known to the person skilled in the art. For example, the DMC compounds can be prepared by direct precipitation, by the “incipient wetness” method, by preparation of a precursor phase and subsequent recrystallization.

The DMC compounds can be used as powder, paste or suspension or can be converted to a molding, introduced into moldings, foams or the like or applied to moldings, foams or the like.

The DMC catalyst concentration used for the alkoxylation in step a), based on the crude polyetherol, is typically less than 2000 ppm, preferably less than 1000 ppm, in particular less than 500 ppm, particularly preferably less than 100 ppm, for example less than 50 ppm.

The addition reaction in step a) under DMC catalysis is carried out at temperatures of about 90 to 240° C., preferably from 100 to 160° C., in a closed vessel. The alkylene oxide is added to the reaction mixture under the vapor pressure of the alkylene oxide mixture prevailing at the chosen reaction temperature and the vapor pressure of the inert gas optionally present (preferably nitrogen).

If an alkylene oxide mixture is used in step a), crude polyetherols in which the various alkylene oxide building blocks are virtually randomly distributed are formed. Variations in the distribution of the building blocks along the polyether chain result from different reaction rates of the components and can also be achieved randomly by continuous feeding of an alkylene oxide mixture of program-controlled composition. If the various alkylene oxides are reacted in succession, polyether chains having block-like distribution of the alkylene oxide building blocks are obtained.

The length of the polyether chains varies randomly within the reaction product about a mean value of the stoichiometric values resulting substantially from the amount added.

The amine-catalyzed process can be carried out analogously to the DMC-catalyzed process, an amine catalyst being used instead of a DMC catalyst.

Catalysts used are basic compounds, such as tertiary amines. Examples of amine catalysts are piperazine, derivatives such as 1,4-dimethylpiperazine, N-hydroxyethylpiperazine, 1,3,5-tris(dimethylaminopropyl)hexahydrotriazine, and/or N,N-dimethylcyclohexylamine, dimethylbenzylamine 2,2′-bis(2-ethyl-2-azobicyclo ether) 1,8-diazabicyclo[5.4.0]undec-7-ene and morpholine derivatives, such as 4-methyl- and 4-ethylmorpholine, and 2,2-dimorpholinoethyl ether, imidazole derivatives, such as 1-methyl- and 1,2-dimethylimidazoles, N-(3-aminopropyl)imidazole, diazobicyclooctane, triethylamine, dimethylaminopropylamine, diethylaminoethylamine, trimethylamine (TMA), tributylamine, triethylamine (TEA), dimethylethanolamine (DMEOA), dimethylcyclohexylamine (DMCHA), imidazole and substituted imidazole derivatives, preferably dimethylethanolamine. Said catalysts can be used individually or as a mixture with one another. The catalyst concentration, based on the total mass of the polyol, may be from 0.01 to 10% by weight. Catalyst concentrations of 0.05-5% by weight, particularly preferably of 0.1-2% by weight, based in each case on the total amount of the polyol, are preferred.

The amine-catalyzed process is carried out at temperatures of from 50 to 180° C. The pressure in the reactor is chosen so that the alkylene oxides remain to a large extent liquid.

In a particular embodiment, the crude polyetherol prepared in step a) by a continuous process is used directly in step b) for removing low molecular weight byproducts.

According to the invention, it is possible to add a stabilizer to the reaction mixture or to one of the components before or after the reaction according to step (a). This can prevent the formation of undesired byproducts owing to oxidation processes.

In the context of the present invention, in principle all stabilizers known to the person skilled in the art can be used.

These components comprise antioxidants, free-radical scavengers, peroxide decomposers, synergistic agents and metal deactivators.

Antioxidants used are, for example, sterically hindered phenols and aromatic amines.

For removing low molecular weight byproducts from the crude polyetherol prepared in step a), said crude polyetherol is treated in step b) with a stripping gas by the process according to the invention in a column having internals. In a preferred embodiment, step b) is carried out continuously. In a particularly preferred embodiment, both step a) and step b) are effected in a manner such that a continuous overall process for the preparation of polyetherols results.

For this purpose, the crude polyetherols are stripped via a column having internals under reduced pressure at elevated temperatures. In the context of the present invention, crude polyetherol is understood as meaning polyetherols which are prepared by one of the processes described above in step a) and comprise impurities, such as low molecular weight byproducts.

In the context of the present invention, stripping is understood as meaning a process in which the low molecular weight byproducts are removed from the crude polyetherols by passing a stripping gas through them, and are transferred to the stripping gas. Steam and/or inert gas are used as the stripping gas. Nitrogen-containing gas mixtures, in particular nitrogen, are preferred as inert gas. In a preferred embodiment, a steam-containing stripping gas, such as steam or a mixture of steam and nitrogen, is used. In a particularly preferred embodiment, steam is used as the stripping gas.

Desorption—also referred to as stripping—is understood as meaning the selective passage of dissolved liquid components into the “inert” gas phase because of partition equilibria between gas phase and liquid phase. The stripping is a special form of distillation. It differs therefrom in that the second phase required for separation of substances on the basis of partition equilibria is not produced by evaporation but is added as an assistant (stripping gas). One possibility for carrying out a desorption is the expulsion of the component to be separated off in the inert gas stream or steam stream. The carrier gas is fed countercurrent to the laden solvent. The component to be separated off migrates from the liquid phase into the gas phase. In the case of inert gas as carrier gas, the partial pressure of the component to be separated off in the gas phase is kept low by continuously added inert gas.

For this purpose, the crude polyetherol is passed countercurrent, i.e. against the direction of flow of the stripping gas, through a column having internals. The column is operated with an irrigation density of from 0.5 to 20 m³/m²*h, preferably with from 2 to 15 m³/m²*h. The irrigation density thus indicates the volume of crude polyol used per hour and cross section of the column. In the stripping of rigid foam polyetherols, an irrigation density of from 10 to 15 m³/m²*h is particularly preferred. In the stripping of flexible foam polyetherols, an irrigation density of from 5 to 10 m³/m²*h is particularly preferred.

The stripping is carried out at temperatures in the range from 20 to 300° C., preferably at from 80 to 200° C., and particularly preferably at from 100 to 160° C. The column is operated at a pressure of from 2 to 300 mbar (absolute), preferably at a pressure of from 5 to 80 mbar (absolute) and particularly preferably at a pressure of from 8 to 60 mbar (absolute). On stripping with H₂O, the vapor pressure level (p) is preferably chosen so that the water content in the end product is from 0.01 to 0.2%, preferably from 0.05 to 0.15%.

In the case of higher water contents, a separate drying step can be effected subsequently, in which the polyol is further dried at temperatures of, preferably, from 100 to 160° C. under a reduced pressure of 2-300 mbar in a period of from 5 minutes to 2 hours.

The stripping gas is fed in in amounts of from 1 to 30 m³ (S.T.P.) (cubic meters under standard conditions) per metric ton of polyetherol, preferably from 2 to 20 m³ (S.T.P.) per metric ton of polyetherol, particularly preferably from 3 to 10 m³ (S.T.P.) per metric ton of polyetherol and most preferably 4-6 m³ (S.T.P.) per metric ton of polyetherol.

For polyols having viscosities of <5000 mPa·s (25° C.), the ratio of polyol to stripping gas is in general 0.1-10 mol of polyol/mole of stripping gas, preferably from 0.2 to 5 mol of polyol/mole of stripping gas, particularly preferably from 0.3 to 3 mol of polyol/mole of stripping gas and most preferably 0.4-2 mol of polyol/mole of stripping gas.

For polyols having viscosities of >5000 mPa·s, the ratio of polyol to stripping gas is 0.4-30 mol of polyol/mole of stripping gas, preferably from 0.7 to 25 mol of polyol/mole of stripping gas, particularly preferably from 1 to 20 mol of polyol/mole of stripping gas and most preferably 1.3-15 mol of polyol/mole of stripping gas.

Suitable residence times of the crude polyetherol in the column are in the range from 1 to 100 minutes, preferably in the range from 5 to 40 minutes.

In the context of the present invention, column having internals is understood as meaning columns which have internals with separation activity. In principle, all known internals, in particular trays, random packings or structured packings, can be used for this purpose.

Suitable internals are customary internals, such as commercially available trays, random packings or structured packings, for example bubble trays, tunnel trays, valve trays, sieve trays, dual flow trays and grid trays, Pall rings®, Berl® saddles, wire mesh rings, Raschig rings®, Interlocks® saddles, Interpack® packings and Intos® packings, but also structured packings, such as Sulzer Mellapak and Mellapakplus, Sulzer-Optiflow® , Kühni-Romopak®, MontzA3-500® fabric packings, SulzerBX® fabric packings, Sulzer Mellacarbon, Sulzer Mellagrid, Nuttergrid, gauze packing Type BX, gauze packing BXPlus, gauze packing Type CY, gauze packing Type DX, gauze packing Type EX, Montz-Pak Type B1, Montz-Pak Type BSH, Montz-Pak Type A3, Montz-Pak Type M, Montz-Pak Type MN, MontzPak Type C1, Koch-Glitsch Flexipac and Flexipac HC, Koch-Glitsch Gempak, knitted wire packing ACS and ACSX, Raschig-PAK and Raschig Super-PAK, Kühni Rombopak and Rombopak S.

Packed columns are preferred. These are understood as meaning packed columns which comprise random packings or structured packing elements. Random packings used may be packing elements comprising materials such as steel, stainless steel, copper, carbon, earthenware, porcelain, glass and plastic. Suitable packing elements for columns which comprise random packings are described, for example, in Klaus Sattler, Thermische Trennverfahren, VCH-Verlag, 1995. In a particularly preferred embodiment, packed columns having structured packing elements are used. Structured packing elements are, for example, knitted wire, sheet metal or fabric packings.

Fabric packings are particularly preferred; MontzA3-500® and SulzerBX® fabric packings are especially preferred.

In a further embodiment, the stripping gas used is a steam-containing stripping gas, steam being particularly preferred. Surprisingly, it was found that in spite of the use of steam-containing gas mixtures as stripping gas, a drying step for the polyetherols obtained is not necessary in the process according to the invention.

The present invention therefore also relates to a process for removing low molecular weight byproducts from crude polyetherols using a steam-containing stripping gas, in which no drying step for the purified polyetherol is carried out after the stripping.

In addition, the present invention also relates to the polyetherols obtainable by the process according to the invention. The present invention therefore also relates to a polyetherol obtainable by a process at least comprising the following steps
a) reaction of at least one starter compound with at least one alkylene oxide and a catalyst to give a crude polyetherol and
b) removal of low molecular weight byproducts from the crude polyetherol from step a), wherein the crude polyetherol is treated with a stripping gas in a column having internals.

The polyetherols obtainable by the process according to the invention are distinguished in particular by a small proportion of impurities. They have little odor and low FOG (fogging) and VOC (volatile organic compounds) values. In particular, the polyetherols according to the invention have low residual alkylene oxide values, preferably less than or equal to 100 ppm, more preferably less than or equal to 50 ppm and particularly preferably less than or equal to 20 ppm, based in each case on the polyetherol.

Owing to the small proportions of impurities, the flexible foam polyetherols prepared according to the invention are suitable in particular for the preparation of polyurethanes for the automotive and furniture industry.

The rigid foam polyetherols are used predominantly in insulation technology, in household appliances and in the construction industry. The present invention therefore also relates to the use of a polyetherol obtainable by the process according to the invention for the synthesis of polyurethanes.

The polyetherols prepared according to the invention are suitable in particular for the production of polyurethane foams, polyurethane cast skins and elastomers. Preferably, the polyetherols prepared according to the invention are used for the synthesis of polyurethane flexible foam. Said polyurethane flexible foam may be slabstock flexible foams or molded flexible foams. In a further embodiment, the present invention therefore relates to the use of a polyetherol obtainable by a process according to the invention or of a polyetherol according to the invention for the synthesis of polyurethanes, the polyurethane being a polyurethane flexible foam.

Among polyurethane foams, in particular foams which are used in the automotive and furniture industry are preferred. Such polyurethanes are suitable, for example, for the production of moldings, in particular moldings made of flexible polyurethane slabstock foam. What is advantageous here is the low content of impurities, since no troublesome odors thus occur or which may be released from the shaped flexible foam article. In addition, the VOC and FOG values are low.

Moldings according to the invention are, for example, mattresses, cushions, shaped articles for the automotive industry and upholstered furniture.

The invention is explained in more detail by the examples, without limiting it thereto.

### EXAMPLE 1

### Preparation of a DMC-Catalyzed Flexible Foam Polyetherol (polyol I)

3200 g of a glycerol-started propoxylate worked up with phosphoric acid and having an OH number of 298 mg KOH/g were mixed with 44 g of a 4.53% strength DMC catalyst suspension (corresponding to 100 ppm of DMC catalyst, based on the product to be prepared) in a 20 l stirred vessel reactor and dewatered at 120° C. and a reduced pressure of 40 mbar till the water content was below 200 ppm. Thereafter, about 400 g of 1,2-propylene oxide (PO) were metered in and time was allowed for the reaction to start, which was detectable from a brief temperature increase and a rapid drop in the reactor pressure. Thereafter, 16450 g of a mixture of 14910 g of PO and 1940 g of ethylene oxide (EO) were fed in at the same temperature over a period of 2.5 h. After a constant reactor pressure had been reached, unreacted monomer and other volatile constituents were distilled off under reduced pressure and the polyetherol was discharged.

The polyetherol (polyol I) obtained has the following characteristics:

OH number (OHN): 48.8 mg KOH/g

Average molecular weight: 3420 g/mol

Acid number: 0.013 mg KOH/g

Water content: 0.011%

Viscosity: (25° C.) 566 mPa·s

### EXAMPLE 2

### Preparation of an Amine-Catalyzed Rigid Foam Polyetherol (Polyol II)

4700 g of sugar and 1450 g of glycerol are initially taken in a 20 l stirred reactor and the reactor is evacuated. The vacuum is broken with nitrogen, 200 g of dimethylethanolamine are metered in and the mixture is heated to 105° C. After the reaction temperature has been reached, 13500 g of PO are metered over 10 h such that the pressure does not exceed 7 bar. After a constant reactor pressure had been reached, unreacted monomer and other volatile constituents were distilled off under reduced pressure and the polyetherol was discharged.

The polyetherol (polyol II) obtained has the following characteristics:

OHN: 450 mg KOH/g

Viscosity: (25° C.) 19800 mPa·s

Average molecular weight: 630 g/mol

### EXAMPLE 3

The DMC-catalyzed polyetherol (polyol I) was stripped in a laboratory apparatus. The experimental column had a diameter of 0.05 m and possessed fabric packing of the type Montz A3-500 over 7 0.5 m sections. After each section, the liquid was collected and redistributed. The column jacket was thermostated at 130° C. and the top pressure of the column was 50 mbar absolute. Pure polyetherol I was provided, and 400 ppm of 1,2-propylene oxide (PO) was deliberately added before the experiment. Ethylene oxide was neglected since experience shows that, on maintaining the PO specification, EO too is sufficiently depleted. The target specification was in the range of <5 to <1 ppm by weight of PO at the bottom of the column. With this setup, and the setting of F=0.17 Pâ0.5 and an irrigation density of 7 m³/(m²*h), PO concentrations of <1 ppm by weight of PO were reached in the bottom. The polyol I/steam ratio was 0.95 kmol of polyol I/kmol of steam.

F=F factor or gas loading factor is the product of the gas velocity and the square root of the gas density, the gas velocity being the volume flow rate of the gas divided by the free column cross section.

### EXAMPLE 4

An amine-catalyzed polyetherol (polyol II) was stripped in a laboratory apparatus. The experimental column had a diameter of 0.05 m and possessed Montz A3-500 fabric packing over 8 0.5 m sections. After each section, the liquid was collected and redistributed. The bottom temperature was 110-120° C. and the top pressure of the column was 50 mbar absolute. Pure polyetherol was provided, and 1,2-propylene oxide (PO) was deliberately added before the experiment. The target specification was in the range of <1 ppm by weight of PO at the bottom of the column. The starting concentration was 1000 ppm by weight of PO and 8 kg/h of polyetherol (feed temperature: 125° C. at 5 bar absolute). With this setup, and the setting of F=0.18 Pâ0.5 and a liquid load of 3.73-3.96 m³/(m²*h), PO concentrations of <1 ppm by weight of PO were reached at the bottom. Furthermore, 0.265 kg/h of nitrogen was passed through the column. The polyol II/N₂ ratio was therefore 1.31 mol of polyol II/kmol of N₂ to 1.38 kmol of polyol II/kmol of N₂.

### EXAMPLE 5

An amine-catalyzed polyetherol (polyol II) was stripped in a laboratory apparatus. The experimental column had a diameter of 0.05 m and possessed Montz A3-500 fabric packing over 8 0.5 m sections. After each section, the liquid was collected and redistributed. The bottom temperature was 120-125° C. and the top pressure of the column was 50 mbar absolute. Pure polyetherol was provided, and 1,2-propylene oxide (PO) was deliberately added before the experiment. The target specification was in the range of <1 ppm by weight of PO at the bottom of the column. The starting concentration was 2000 ppm by weight of PO and 12.8 kg/h of polyol (feed temperature: 125° C. at 5 bar absolute). With this setup, and the setting of F=0.17 Pâ0.5 and a liquid load of 6 m³/(m²*h), PO concentrations of <1 ppm by weight of PO were reached at the bottom. Furthermore, 200 g/h of steam were passed through the column. The polyol II/steam ratio was therefore 2.01 of polyol II/mol of steam.

### COMPARATIVE EXAMPLE 1

A bubble column (ID=10 cm) which had a double jacket for thermostating and a ring distributor (d=4 cm) with numerous bores at the bottom for passing in gas was used for the stripping process. The temperature of the bubble column was kept constant by a commercially available thermostat. The pressure in the bubble column was kept constant at 300 mbar by means of a vacuum pump.

For the stripping, 6 kg of the polyol I were pumped under inert conditions into the bubble column rendered inert with nitrogen. Thereafter, the polyol I was heated to the stripping temperature and at the same time the pressure was established in the bubble column. Steam was fed in via the ring distributor, the amount being monitored via a steam meter.

After stripping for 2 hours with a steam flow of 80 g/h, the free PO content was 3 ppm. The polyol I/steam ratio was therefore 0.2 mol of polyol I/mole of steam.

### COMPARATIVE EXAMPLE 2

A bubble column (ID=10 cm) which had a double jacket for thermostating and a ring distributor (d=4 cm) with numerous bores at the bottom for passing in gas was used for the stripping process. The temperature of the bubble column was kept constant by a commercially available thermostat. The pressure in the bubble column was kept constant at 300 mbar by means of a vacuum pump.

For the stripping, 6 kg of the polyol II were pumped under inert conditions into the bubble column rendered inert with nitrogen. Thereafter, the polyetherol was heated to the stripping temperature and at the same time the pressure was established in the bubble column. Nitrogen was fed in via the ring distributor, the amount being monitored via a rotameter.

After stripping for 2 hours with a nitrogen flow of 13 l (S.T.P.)/min, the free PO content was 6 ppm. The polyol II/nitrogen ratio was therefore 16.5 mol of polyol II/mole of nitrogen.

### COMPARATIVE EXAMPLE 3

A bubble column (ID=10 cm) which had a double jacket for thermostating and a ring distributor (d=4 cm) with numerous bores at the bottom for passing in gas was used for the stripping process. The temperature of the bubble column was kept constant by a commercially available thermostat. The pressure in the bubble column was kept constant at 300 mbar by means of a vacuum pump.

For the stripping, 6 kg of the polyol II were pumped under inert conditions into the bubble column rendered inert with nitrogen. Thereafter, the polyetherol 2 was heated to the stripping temperature and at the same time the pressure was established in the bubble column. Steam was fed in via the ring distributor, the amount being monitored via a steam meter.

After stripping for 2 hours with a steam flow of 20 g/h, the free PO content was 12 ppm. The polyol II/steam ratio was therefore 4.3 mol of polyol II/mole of steam.

1. A process for preparing a polyetherol, the process comprising:
a) reacting at least one compound with at least one alkylene oxide and an amine or a DMC catalyst to give a crude polyetherol; and
b) removing at least one low molecular weight byproduct from the crude polyetherol by treating the crude polyetherol with a stripping gas in a column having internals,
wherein the process is carried out continuously.

2. The process of claim 1, wherein the alkylene oxide comprises ethylene oxide, propylene oxide, or a mixture thereof.

3. The process of claim 1, wherein the column is a packed column.

4. The process of claim 3, wherein the packed column comprises a fabric packing.

5. The process of claim 1, wherein the stripping gas is a steam-containing gas mixture and no drying occurs after the removing b) of the byproduct.

6. The process of claim 1, wherein the removing b) of the byproduct is occurs at a temperature in a range from 100 to 160° C.

7. The process of claim 1, wherein the removing b) of the byproduct occurs with an irrigation density of from 0.5 to 20 m³/m²*h.

8. The process of claim 1, wherein the stripping gas is present in amounts of from 1 to 30 m³ (S.T.P.) per metric ton of the polyetherol.

9. The process of claim 1, wherein the removing b) of the byproduct occurs at a pressure of from 2 to 300 mbar (absolute).

10. The process of claim 1, wherein the removing b) of the byproduct occurs at a pressure of from 5 to 80 mbar (absolute).

11. The process of claim 1, wherein the removing b) of the byproduct occurs at a pressure of from 8 to 60 mbar (absolute).

12. The process of claim 2, wherein the column is a packed column.

13. The process of claim 2, wherein the packed column comprises a fabric packing.

14. The process of claim 2, wherein
the stripping gas is a steam-containing gas mixture, and
no drying occurs after the removing b) of the byproduct.

15. The process of claim 2, wherein the removing b) of the byproduct occurs at a temperature in a range from 100 to 160° C.

16. The process of claim 2, wherein the removing b) of the byproduct occurs with an irrigation density of from 0.5 to 20 m³/m²*h.

17. The process of claim 2, wherein the stripping gas is present in amounts of from 1 to 30 m³ (S.T.P.) per metric ton of the polyetherol.

18. The process of claim 2, wherein the removing b) of the byproduct occurs at a pressure of from 2 to 300 mbar (absolute).

19. The process of claim 2, wherein the removing b) of the byproduct occurs at a pressure of from 5 to 80 mbar (absolute).

20. The process of claim 2, wherein the removing b) of the byproduct occurs at a pressure of from 8 to 60 mbar (absolute).
